(19) United States
(12) Patent Application Publication
(10) Pub. No.: US 2013/0098775 A1
(43) Pub. Date: Apr. 25, 2013

(54) Glucose biosensor with improved shelf life

(71) Applicants: Jianhong Pei, Boxborough, MA (US); Andy Vo, Somerville, MA (US); Chung Chang Young, Weston, MA (US)

(72) Inventors: Jianhong Pei, Boxborough, MA (US); Andy Vo, Somerville, MA (US); Chung Chang Young, Weston, MA (US)

(73) Assignee: NOVA BIOMEDICAL CORPORATION, Waltham, MA (US)

(21) Appl. No.: 13/277,687

(22) Filed: Oct. 20, 2011

(51) Int. Cl. G01N 27/327; G01N 33/66

(52) U.S. Cl. 205/777.5; 204/403.04

(57) ABSTRACT

A disposable biosensor having improved long term storage stability including a working electrode and a reference electrode formed on an insulating substrate and a dissolvable reagent layer composition disposed directly on the working electrode where the reagent layer composition contains a glucose-based enzyme, a reagent component selected from a phosphate buffer and a tris reagent, and a redox mediator.

### BACKGROUND OF THE INVENTION

1. Field of the Invention

The present invention relates generally to electrochemical sensors. Particularly, the present invention relates to a biosensor for the detection of glucose in biological fluids.

2. Description of the Prior Art

Diabetes is a metabolic disorder and lifelong disease marked with high levels of sugar in the blood, in which either the pancreas fails to produce insulin or cells lack receptors for insulin. Insulin helps to move glucose from the bloodstream to muscle, fat and liver cells where it can be used as fuel. A lack of insulin or having cells that do not respond to insulin normally will cause excess unused glucose to remain in the bloodstream. The excess sugar destroys small blood vessels, which diminishes the supply of blood to vital organs such as the heart, the brain, and the kidneys. As a result, diabetics often suffer from heart attacks, kidney failures or strokes. According to data from the 2011 National Diabetes Fact Sheet, 25.8 million children and adults in the United States have diabetes. Diabetes is the seventh leading cause of death in the United States and the disease is a major world heath problem.

Blood glucose monitoring has been established as a valuable tool in the management of diabetes. Patients with diabetes are encouraged to monitor their glucose levels, especially to prevent and control hyperglycemia and gestational diabetes, which can help to delay the development and progression of hyperglycemia. A glucose biosensor, which is used in blood glucose monitoring, is an analytical device for detecting the analyte, glucose, in the blood. Although glucose biosensors have been devised based on potentiometry, amperometry, colometry, and colorometry, to date most commercially available biosensors are amperometric biosensors. These biosensors use a redox enzyme as the biological component responsible for the selective recognition of the test species of interest. A biosensor of this type is a relatively small strip of laminated plastic that defines a reaction chamber into which a biological sample such as blood is disposed. An important feature of the biosensor is that it is disposable and only used one time.

The glucose biosensor contains a reagent that can react with the analyte in a sample of the biological fluid. The reagent includes the selective recognition component as well as electron mediators or other substances, which can help facilitate the reaction or help stabilize the reagent. The biological fluid sample is introduced into the sample chamber of the glucose biosensor and the biosensor is connected to a measuring device such as a meter for analysis using the biosensor's electrical contacts. The analyte (glucose) in the sample undergoes an oxidation/reduction reaction inside the reaction chamber while the measuring device applies a biasing potential signal through the electrical contacts of the biosensor. The redox reaction produces an output signal in response to the biasing potential signal. The output signal usually is an electronic signal, such as potential or current, which is measured and correlated with the concentration of the analyte in biological fluid sample. The success in the development of these devices has led to amperometric assays for several biomolecules including glucose, BUN, Lactate, creatinine, uric acid, ketone, and the like.

Because of its high glucose selectivity, glucose oxidoreductase (GOD) (i.e. glucose oxidase) has been widely used as a biological component to make the glucose biosensor. The measurement of glucose using GOD, however, is sensitive to the variation of oxygen concentration in the sample. This is due to the fact that oxygen works as the electron acceptor in the enzyme reaction of GOD. A redox mediator was added to the reagent layer of the glucose biosensor to substitute oxygen as the electron acceptor. The result was to extend the linear range of the response of the glucose biosensor and to allow use of a much lower biasing/working potential. Redox mediators, however, are not as efficient at shuttling electrons with the enzyme as is the oxygen molecule. In fact, any oxygen in the sample solution can compete with the redox mediator for the reduced GOD. Measurements with the mediator/GOD-based biosensor lead to a decrease in signal and an underestimation of glucose concentration with increasing oxygen concentration in the fluid sample. Moreover, biological specimens contain widely varying oxygen levels. For example, oxygen concentration in venous blood is about 30 mmHg while oxygen concentration in capillary blood is about 90 mmHg. For oxygen saturated blood, the oxygen concentration may be as high as to 200 mmHg. This oxygen-related characteristic of the GOD-based biosensor limits the reliability of these types of biosensors.

To overcome the interference resulting from the varying oxygen concentration or so-called “oxygen effect” associated with the use of glucose oxidase, glucose dehydrogenase having pyrroloquinoline quinine (PQQ) co-enzyme, which do not use oxygen as the electron acceptor, was recently used to replace glucose oxidase. This compound is a promising candidate for electrocatalytic oxidation of glucose in an oxygen-insensitive glucose sensor. There are two types of PQQ-GDH; one is intracellular and soluble, while the other is tightly bound to the outer surface of the cytoplasmic membrane. Membrane-bound PQQ-GDH (mPQQ-GDH) has high glucose selectivity, but low solubility. Because of its low solubility, mPQQ-GDH is difficult to use in a biosensor application.

The water soluble PQQ-GDH (sPQQ-GDH) does overcome the problems caused by the oxygen effect of GOD and the low solubility of mPQQ-GDH. However, sPQQ-GDH is not as specific as glucose oxidase. It not only reacts with glucose but also reacts with a variety of monosaccharides and polysaccharides such as galactose, maltose, maltotetraose and maltotetraise. If a glucose biosensor uses a PQQ-GDH as biological recognition component, a falsely high glucose result could be obtained due to the interference from maltose or galactose or the like. In particular, the false response to maltose is a serious issue with the use of peritoneal infusion solution for diabetes because it contains a high concentration of icodextrin, which results in the production of maltose. A false high blood glucose reading could result in a patient being given more insulin than needed. Getting more insulin than needed lowers a patient's blood sugar unnecessarily and may cause a serious reaction, including loss of consciousness.

Unfortunately, glucose biosensors also suffer from the electrochemical oxidation of some endogenous electrochemical active chemicals such as ascorbic acid, bilirubin, uric acid, and the like including some commonly used drugs such as, for example, acetaminophen that are frequently found in biological samples. This also leads to an interference with the electrical current to be detected in amperometric biosensors. One method in diminishing these electroactive interferences is to employ a permselective coating that minimizes the access of these constituents at the electrode surface. Different polymers, multilayers and mixed layers with transport properties based on the charge, size, or polarity have thus been used to block those electroactive interferring species. These coating and/or membrane techniques are difficult to apply in the disposable biosensor industry.

Another important aspect of measuring analytes in whole blood samples is the effect of hematocrit. Whole blood samples generally have a hematocrit concentration in the range from about 20% to about 60%, with about 40% being the average. Hematocrit is the volume of red blood cells expressed as a percentage of volume of red blood cells in the whole blood sample. Red blood cells can block the diffusion of analyte and/or mediator to one or more electrodes of the biosensor, thus affecting the output signal and the accuracy of the measurement results.

As the above description confirms, glucose biosensors are intricate, complicated devices relying on the chemistry of the reagent layer and the chemical reactions involved to obtained a desired result. A minor change in sample composition can have unexpected results depending on the reagent layer composition and structure.

Recently, a novel flavin adenine dinucleotide (referred to as FAD) dependent glucose dehydrogenase (GDH-FAD) has been made from Aspergillusterreus. FAD-GDH is completely insensitive to oxygen and has high substrate selectivity. Neither the oxygen concentration nor the sugars such as maltose, galactose, etc. interfere with the measurement of glucose. This novel enzyme is a very promising candidate for making a reliable and interference-free glucose biosensor. One such glucose biosensor is disclosed in US Patent Application Publication No. 2009/0065356. The biosensor includes a reagent layer that includes an enzyme GDH-FAD, a redox mediator, and an additive agent which is one of an organic acid or organic acid salt having at least one carboxyl group in a molecule, an organic acid or organic acid salt having at least one amino group or carbonyl group in a molecule, a sugar alcohol, and a solubilized protein, or a combination thereof.

### SUMMARY OF THE INVENTION

Despite the improvements made in the development of the glucose biosensor, one of the major problems with biosensors is their long term stability when stored. Biosensors are typically manufactured at some time prior to their use. Between their manufacture and use, the biosensors are stored. During this storage period, the biosensors may be exposed to various environmental conditions where heat and moisture exist. Particularly under high temperature and humidity conditions, a reduction reaction occurs between the electron transfer agent/redox mediator and a portion of enzyme protein or hydrophilic polymer, which are typically included in a reagent layer composition. This leads to the generation of background electric current. The enzyme may also lose activity caused by various environmental conditions, which results in a decrease of current. As the value of the background electric current increases or the enzyme derived current decreases (or both) with time, the biosensor performance is deteriorated.

One way to solve this problem is to eliminate moisture and prevent the deterioration of the sensor performance by enclosing a desiccant such as silica gel or activated alumina into a biosensor preservation container which employs a molded container of resin and an aluminum seal. However, it is extremely difficult to keep the preservation container free of moisture penetration over a long period of time. Also, the user may unintentionally leave the cover of the vial open exposing the biosensors to ambient room humidity. The reduction reaction between a portion of enzyme protein or hydrophilic polymer and the electron transfer agent/redox mediator is promoted when only a slight amount of moisture exists. Therefore, it is extremely difficult to effectively suppress the increase in the background electric current with time or the decrease in enzyme activity.

To reduce the environmental effect, it has been reported that the use of buffering agent of polycarboxylic acids can help stabilize the GDH-FAD enzyme reaction. By polycarboxylic acids is meant that the buffering agents include two or more carboxylic acid functional moieties, where the number of different carboxylic acid functional moieties may range from about 2 to about 10, e.g., from about 2 to about 8, including from about 2 to about 6. The carboxylic acid groups or functional moieties of the subject buffering agents may be attached to a number of different structures, including aliphatic, alicyclic, aromatic and heterocyclic structures. The presence of more than one carboxylic acid group can have the beneficial effect of providing at least one pKa value for the buffer in the desired range.

An organic acid or organic acid salt having at least one carboxyl group in the molecule also functions to prevent the aforementioned reduction of the electron transfer agent/redox mediator. The organic acid or organic acid salt having at least one carboxyl group in a molecule may be aliphatic carboxylic acid, carbocyclic carboxylic acid, heterocyclic carboxylic acid, or their salts. The aliphatic carboxylic acid may be malonic acid, succinic acid, glutaric acid, adipic acid, maleic acid, fumaric acid, or their salts. The carbocyclic carboxylic acid may be acidum benzoicum, phthalic acid, isophthalic acid, terephthalic acid, or their salts, and the same effects as described above can be achieved by using these materials. The heterocyclic carboxylic acid may be 2-furoic acid, nicotinic acid, isonicotinic acid, or their salts, and the same effects as described above can be achieved by using these materials. Besides the above-described aliphatic or carbocyclic carboxylic acid, and carboxylic acid or carboxylate salt having a heterocyclic ring, there may be adopted, for example, malic acid, oxaloacetic acid, citric acid, ketoglutaric acid and their salts in which functional groups of the carboxylic acid or carboxylate salt are partially replaced with other functional groups, with the same effect as described above.

It is an object of the present invention to provide glucose biosensor having long term storage stability. It is another object of the present invention to provide a glucose biosensor having long term storage stability without the addition of carboxylic acid based buffering agents.

The present invention achieves these and other objectives by providing a glucose biosensor that has long term storage stability without the use of carboxylic acid based buffering agents.

In one embodiment, a disposable biosensor having improved long term stability includes a working electrode and a reference electrode formed on an insulating substrate and a dissolvable reagent layer composition disposed directly on the working electrode where the reagent layer composition contains a glucose-based enzyme, one of tris reagents or phosphate reagents, a redox mediator and a polymer binder.

In another embodiment, the glucose-based enzyme is glucose dehydrogenase having flavin adenine dinucleotide as a coenzyme.

In a further embodiment of the present invention, the glucose-based enzyme is one of glucose oxidase, PQQ-glucose dehydrogenase or NAD-glucose dehydrogenase.

In another embodiment, the working electrode is made of gold, gold/tin oxide, platinum, or palladium.

In a further embodiment, the tris reagents or phosphate reagents have a pH in the range of 4.0 to 9.0.

In yet another embodiment, the tris reagent is the acid of tris(hydroxymethyl)aminomethane or salts thereof.

In another embodiment of the biosensor, the tris reagent is selected from the group consisting of tris(hydroxymethyl)aminomethane acetate, tris(hydroxymethyl)aminomethane benzoate, tris(hydroxymethyl)aminomethane carbonate, tris(hydroxymethyl)aminomethane citrate, tris(hydroxymethyl)aminomethane hydrochloride, tris(hydroxymethyl)aminomethane maleate, tris(hydroxymethyl)aminomethane nitrate, tris(hydroxymethyl)aminomethane oxalate, tris(hydroxymethyl)aminomethane phosphate, tris(hydroxymethyl)aminomethane succinate, and tris(hydroxymethyl)aminomethane sulfate.

In another embodiment of the biosensor, the phosphate reagent is selected from the group consisting of phosphoric acid, monobasic phosphate, dibasic phosphate, and tribasic phosphate.

In a further embodiment of the biosensor of the present invention, further comprising one or more of a surfactant and a bulking agent.

In another embodiment of the biosensor of the present invention, glucose-based enzyme is selected from the group consisting of glucose oxidase, PQQ-glucose dehydrogenase, NAD-glucose dehydrogenase, and glucose dehydrogenase having flavin adenine dinucleotide as a coenzyme, the polymer binder is one or more selected from the group consisting of polyethylene oxide (PEO), polyethylene glycol, polyvinylpyrrolidone, starch, methylcellulose, hydroxypropylcellulose, polyvinyl alcohol (PVA), carboxy methyl cellulose (CMC), fish gelatin, and polyamino acids, the redox mediator is selected from the group consisting of potassium ferricyanide, potassium ferrocyanide, hexaamineruthenium (III) chloride, dimethylferrocene, ferricinium, 1,1′-ferrocene dicarboxylic, Meldola's blue, TCNQ, hydroquinone, dichlorophenoliondophenol, p-benoquinone, o-phenylenediamine, 3,4-dihydroxybenzaldehyde, and 1,10-phenanthroline quinine, the surfactant is selected from the group consisting of anionic, cationic, non-ionic, and zwitterionic detergents, and the bulking agent is one or more selected from the group consisting of trehalose, galactose, sucrose, lactose, mannitol, mannose, fructose, lactitol, sorbitol, xylitol, nicotinamide, maltose, and starch.

In a further embodiment of the biosensor of the present invention, the biosensor has a laminated body having an inlet at a first end, a plurality of electrical contacts at an opposite second end, a substantially flat sample chamber in communication between the inlet and a vent opening, a working electrode and a reference electrode within the sample chamber.

In still another embodiment of the present invention, there is disclosed a reagent layer composition for use in a biosensor for amperometric detection of glucose in biological fluids where the biosensor has an improved shelf life. The reagent layer composition includes a glucose-based enzyme, a tris reagent or phosphate reagent, a redox mediator, and a polymer binder where the glucose-based enzyme reacts with a specific compound in a sample solution enabling the biosensor to measure a concentration of the specific compound in the sample.

In another embodiment, the glucose-based enzyme is glucose dehydrogenase having flavin adenine dinucleotide as a coenzyme.

In a further embodiment of the present invention, the glucose-based enzyme is one of glucose oxidase, PQQ-glucose dehydrogenase or NAD-glucose dehydrogenase.

In another embodiment of the reagent layer composition, the tris reagent or phosphate reagent has a pH in the range of 4.0-9.0.

In a further embodiment of the reagent layer composition, the tris reagent is a predefined mixture of tris(hydroxymethyl)aminomethane and tris(hydroxymethyl)aminomethane hydrochloride or acid or base form.

In yet another embodiment of the reagent layer composition, the tris reagent is tris(hydroxymethyl)aminomethane or salts thereof.

In still a further embodiment of the reagent layer composition, the tris reagent salt is selected from the group consisting of tris(hydroxymethyl)aminomethane acetate, tris(hydroxymethyl)aminomethane benzoate, tris(hydroxymethyl)aminomethane carbonate, tris(hydroxymethyl)aminomethane citrate, tris(hydroxymethyl)aminomethane maleate, tris(hydroxymethyl)aminomethane nitrate, tris(hydroxymethyl)aminomethane oxalate, tris(hydroxymethyl)aminomethane phosphate, tris(hydroxymethyl)aminomethane succinate, and tris(hydroxymethyl)aminomethane sulfate.

In another embodiment of the reagent layer composition, the reagent layer composition further includes one or more of a surfactant and a bulking agent.

In a further embodiment of the reagent layer composition, the polymer binder is one or more selected from the group consisting of polyethylene oxide (PEO), polyethylene glycol, polyvinylpyrrolidone, starch, methylcellulose, hydroxypropylcellulose, polyvinyl alcohol (PVA), carboxy methyl cellulose (CMC), fish gelatin, and polyamino acids, the redox mediator is selected from the group consisting of potassium ferricyanide, potassium ferrocyanide, hexaamineruthenium (III) chloride, dimethylferrocene, ferricinium, 1,1′-ferrocene dicarboxylic, Meldola's blue, TCNQ, hydroquinone, dichlorophenoliondophenol, p-benoquinone, o-phenylenediamine, 3,4-dihydroxybenzaldehyde, and 1,10-phenanthroline quinine, the surfactant is selected from the group consisting of anionic, cationic, non-ionic, and zwitterionic detergents, and the bulking agent is one or more selected from the group consisting of trehalose, galactose, sucrose, lactose, mannitol, mannose, fructose, lactitol, sorbitol, xylitol, nicotinamide, maltose, and starch.

In another embodiment of the present invention, a disposable biosensor having improved long term storage stability that includes a working electrode and a reference electrode formed on an insulating substrate where the working electrode is made of palladium, and a dissolvable reagent layer composition disposed directly on the working electrode wherein the reagent layer composition contains a glucose-based enzyme, a buffer, a redox mediator and a polymer binder.

In a further embodiment of the present invention, there is disclosed a method of providing a biosensor with improved long term storage stability that includes incorporating either a phosphate reagent or a tris reagent in the reagent layer composition used to form the working electrode.

In another embodiment of the present invention, there is disclosed a method of providing a biosensor with improved long term storage stability that includes incorporating palladium as the electrically-conductive substrate on a dielectric material for forming the working electrode.

### BRIEF DESCRIPTION OF THE DRAWINGS

FIG. 1 is a perspective view of one embodiment of the present invention showing the test strip.

FIG. 2 is an exploded view of the embodiment in FIG. 1 showing the four component layers of the test strip.

FIG. 3 is a perspective view of another embodiment of the present invention showing the test strip.

FIG. 4 is an exploded view of the embodiment in FIG. 3 showing the three component layers of the test strip.

### DETAILED DESCRIPTION OF THE PREFERRED EMBODIMENT

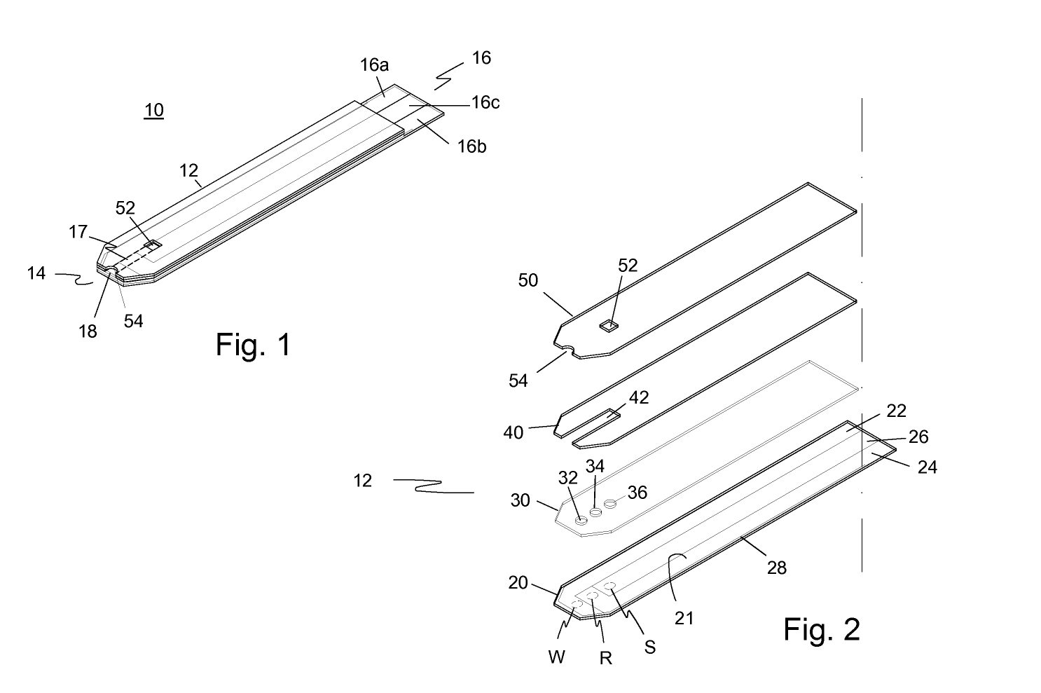

The preferred embodiment(s) of the present invention is illustrated in FIGS. 1-4. FIG. 1 illustrates one embodiment of the present invention. The glucose sensor 10 has a laminated body 12, a fluid sampling end 14, an electrical contact end 16, and a vent opening 52. Fluid sampling end 14 includes a sample chamber 17 between a sample inlet 18 and vent opening 52. Electrical contact end 16 has three discrete conductive contacts 16a, 16b and 16c.

Turning now to FIG. 2, laminated body 12 includes a base layer 20, a reagent holding layer 30, a channel forming layer 40, and a cover 50. All layers of laminated body 12 are made of a dielectric material, preferably plastic. Examples of a preferred dielectric material are polyvinyl chloride, polycarbonate, polysulfone, nylon, polyurethane, cellulose nitrate, cellulose propionate, cellulose acetate, cellulose acetate butyrate, polyester, polyimide, polypropylene, polyethylene, and polystyrene.

Base layer 20 has an electrically-conductive layer 21 on which is delineated three electrically-conductive paths 22, 24 and 26. The electrically-conductive paths 22, 24, 26 may be formed by scribing or scoring conductive layer 21. In the alternative, base layer 20 may be a dielectric material on which conductive paths 22, 24, 26 are silk screened.

Scribing or scoring of conductive layer 21 may be done by mechanically scribing the conductive layer 21 sufficiently to create the three independent, electrically-conductive paths 22, 24, 26. The preferred scribing or scoring method of the present invention is done by using a laser. Conductive layer 21 may be made of any electrically conductive material such as, for example, gold, tin oxide/gold, palladium, platinum, silver other noble metals or their oxides, or carbon film compositions. The preferred electrically conductive material is gold, palladium or tin oxide/gold. The more preferred material for the electrically-conductive layer 21 is palladium and the preferred base layer/electrically-conductive layer material combination is a palladium coated polyester film. An additional scoring line 28 (enlarged and not to scale; for illustrative purposes only) may be made along the outer edge of base layer 20 in order to avoid possible static problems that may give rise to a noisy signal. It should be understood, however, that scoring line 28 is not necessary to the functionality of sensor 10. The conductive layer 21 may also be done by screen printed of carbon ink or carbon paste.

Reagent holding layer 30 has two, but preferably three, electrode openings; a first electrode opening 32 which exposes a portion of first electrically-conductive path 22; a second electrode opening 34 which exposes a portion of second electrically-conductive path 24; and a third electrode opening 36 which exposes a portion of third electrically-conductive path 26. Reagent holding layer 30 is made of a plastic material, preferably a medical grade, one-sided adhesive tape available from Adhesive Research, Inc., of Glen Rock, Pa. or Global Instrument Corporation (GIC) (Taiwan). Acceptable thicknesses of the tape for use in the present invention are in the range of about 0.001 in. (0.025 mm) to about 0.005 in. (0.13 mm). The preferred thickness is about 0.003 in. (0.075 mm). It should be understood that the use of a tape is not required. Reagent holding layer 30 may be made from a plastic sheet and may be coated with a pressure sensitive adhesive, a photopolymer, ultrasonically-bonded to base layer 20, or silk-screened onto the base layer 20 to achieve the same results as using the polyester tape mentioned.

The electrode openings define electrode wells. In a two electrode system, there is a working electrode (W) and a reference/counter electrode (R). In the two electrode system, measurement values may only be corrected for hematocrit but not for interferents (measurement signal interference), provided the required electronic circuits are incorporated into the electronic measuring device, i.e. the meter. This is more clearly explained later. In a three electrode system, there is included an optional substrate electrode (S) in addition to the working electrode W and the reference/counter electrode R. In the three electrode system, measurement values may be corrected for both hematocrit and interferents.

Preferably, electrode well W is loaded with a reagent layer composition that contains an enzyme, an electron/redox mediator, a buffer, and a polymer binder. The enzyme is preferably FAD-GDH although other glucose-based enzymes may be used as disclosed below. Substrate electrode well S is loaded with reagent layer composition that has a similar chemistry to the reagent layer composition in working electrode well W, but without an enzyme. One or more chemical components such as additional polymers, stabilizers, surfactants, and bulking agents may be optionally included in the reagent layer composition. In the preferred embodiment, reference electrode well R is loaded with an electron mediator. Preferably, the electron mediator is potassium ferricyanide, or potassium ferrocyanide or a mixture of potassium ferricyanide and potassium ferrocyanide. In the alternative, the reference electrode (electrode well R) may be loaded with a Ag/AgCl layer (e.g., by applying Ag/AgCl ink or by sputter-coating (a) a Ag layer followed by chloridizing the Ag or (b) a Ag/AgCl layer) or other reference electrode materials that can function properly as a reference electrode.

The size of the reagent holding openings in the 4-layer embodiment is preferred to be made as small as possible in order to make the sample chamber of the biosensor sensor as short as possible. This, in turn, minimizes the volume of sample required for each test measurement. It is noted that the positional arrangement of the electrodes in the channel/sample chamber is not critical for obtaining usable results from the glucose biosensor. The electrodes are each in electric contact with separate electrically-conductive paths. The separate electrically-conductive paths terminate and are exposed for making an electrical connection to a reading device on the end opposite from the sample inlet 18 of laminated body 12.

Channel forming layer 40 has a channel 42 located at the fluid sampling end 14. The length of channel 42 is such that when channel forming layer 40 is laminated to reagent holding layer 30, electrode areas W, R and S are within the space defined by channel 42. The length, width and thickness of the channel 42 define the sample chamber and the capillary chamber volume. Channel forming layer 40 is made of a plastic material, preferably a medical grade, double-sided pressure-sensitive adhesive tape available from Adhesive Research, Inc., of Glen Rock, Pa. or Global Instrument Corporation (Taiwan). The thickness of the tape is preferably in the range of about 0.001 in. (0.025 mm) to about 0.010 in. (0.25 mm). Channel 42 may be made with a laser or by die-cutting (the preferred method). The length of channel 42 is about 0.200 in. (5.08 mm) to about 0.250 in. (6.35 mm), the width is about 0.05 in. (1.27 mm) to about 0.07 in. (1.778 mm). It should be understood that the thickness and the size of channel 42 are not critical.

Cover 50, which is laminated to channel forming layer 40, has vent opening 52 spaced from fluid sampling end 14 of sensor 10 to ensure that the sample in the sample chamber 17 will completely cover electrode areas W, R and S when the sample is introduced into sample inlet 18. Vent opening 52 is positioned in cover 50 so that it will expose a portion of and partially overlay channel notch 42 at or near the closed end of the channel 42. Vent opening 52 may be any shape but is illustrated as a rectangle having dimensions of about 0.08 in. (2 mm) by about 0.035 in. (0.9 mm). The preferred material for cover 50 is a polyester film. Transparency films from 3M or from GIC may be used to form cover 50. Cover 50 may optionally include an inlet notch 54 to prevent an inadvertent occlusion of sample inlet 18 (which can prevent the proper transfer of the sample fluid to the electrodes) when applying a blood sample to sample chamber 17.

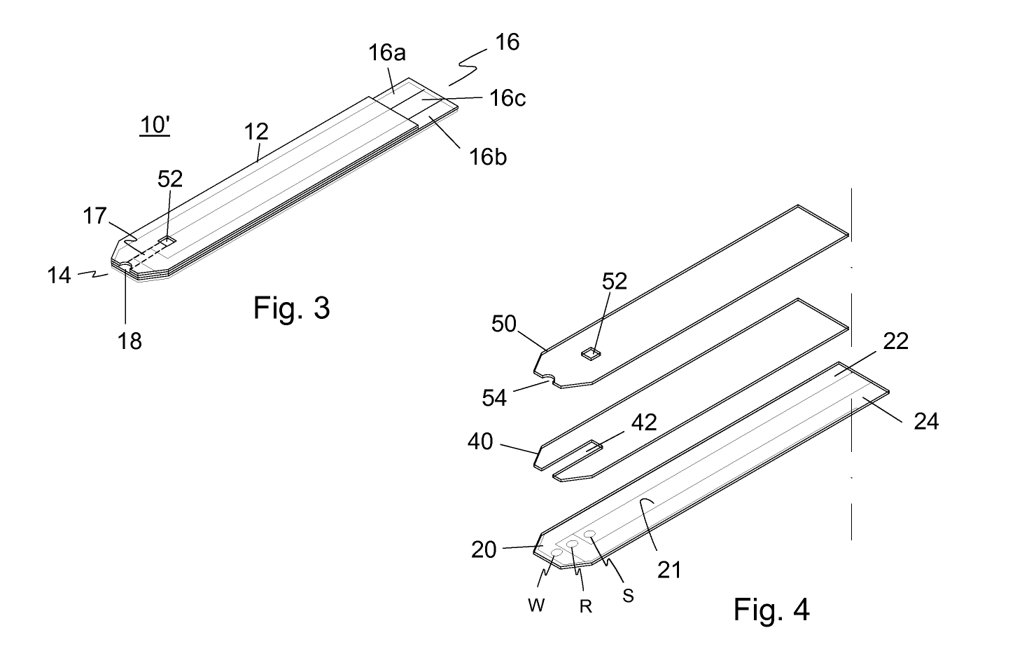

FIG. 3 illustrates a 3-layer embodiment of biosensor 10′. Like the 4-layer embodiment, biosensor 10′ has a laminated body 12, a fluid sampling end 14, an electrical contact end 16, and a vent opening 52. Fluid sampling end 14 includes a sample chamber 17 between a sample inlet 18 and a vent opening 52. Electrical contact end 16 has three discrete conductive contacts 16a, 16b and 16c. As can be seen from FIG. 4, laminated body 12 includes a base layer 20, a channel forming layer 40, and a cover 50. As noted earlier, all layers of laminated body 12 are made of a dielectric material, preferably plastic. Unlike the 4-layer embodiment, there is no separate reagent holding layer in the 3-layer embodiment. Channel forming layer 40 also delineates the area in which a pre-determined amount of reagent layer compositions are disposed. Drops/droplets of the reagent layer compositions are disposed into the reagent channel formed by the channel forming layer containing electrically-conductive paths. The drops/droplets are then dried forming a solidified but dissolvable reagent coating.

Although the preferred embodiments disclosed encompass either a 4-layer configuration of a 3-layer configuration containing three electrically-conductive paths, it is readily recognized by those of ordinary skill in the art that these same configurations may be configured to contain only two electrically-conductive paths and to form a two electrode biosensor.

It should be understood that the electrically-conductive conduit paths in any of the embodiments disclosed herein may be made from any non-corroding metal. Carbon deposits such as for example carbon paste or carbon ink may also be used as the conduit paths, all as is well known by those of ordinary skill in the art. The test reagents may also be deposited on the electrode in one or more ink-based layers. The reagents may be mixed with carbon ink or carbon paste and be screen-printed onto electrodes.

Chemical Reagents

Enzyme

The glucose biosensor of the present invention includes at least chemical agents in the working electrode W that can specifically react with glucose in the sample solution. The chemical agents are preferably glucose related enzymes, including, but not limited to, glucose oxidase, PQQ-GDH, NAD-GDH, and FAD-GDH. The preferred glucose-related enzyme is FAD-GDH. FAD-GDH is a very high substrate-selective enzyme and also completely insensitive to oxygen.

Mediator

A mediator must be included in the reagent layer composition or within the sample chamber when fabricating the working electrode of the glucose biosensor. It is preferable to use an electron (redox) mediator in its oxidized form. The redox mediator may be selected from, but not limited to, potassium ferricyanide, potassium ferrocyanide, hexaamineruthenium (III) chloride, dimethylferrocene, ferricinium, 1,1′-ferrocene dicarboxylic, Meldola's blue, TCNQ, hydroquinone, dichlorophenoliondophenol, p-benoquinone, o-phenylenediamine, 3,4-dihydroxybenzaldehyde, 1,10-phenanthroline quinine. Of these electron mediators, potassium ferricyanide is the mediator of choice in the present invention. The concentration of potassium ferricyanide in the reagent layer composition is preferably 1% to 15% (w/w).

Polymer Binder

The polymer used as the binder in the reagent layer composition should be sufficiently water-soluble and should also be capable of stabilizing and binding all other chemicals in the reagent layer composition to the conductive surface layer in the electrode area. Suitable polymers include, but are not limited to, polyethylene oxide (PEO), polyethylene glycol, polyvinylpyrrolidone, starch, methylcellulose, hydroxypropylcellulose, polyvinyl alcohol (PVA), carboxy methyl cellulose (CMC), fish gelatin, and polyamino acids. The reagent binder may be a single polymer or a combination of polymers. The molecular weight of the PEO ranges from thousands to millions and is available from Scientific Polymer Products, NY, USA. The concentration of PEO in the reagent layer composition is preferably about 0.04% (w/w) to about 2%. Methylcellulose, which is available under the brand name of Methocel 60 HG (Cat. No. 64655, Fluka Chemicals, Milwaukee, Wis., USA) has a concentration in the reagent layer composition preferably in the range of about 0.02% (w/w) to about 5%.

Surfactant

A surfactant may be optionally included in the reagent layer composition to facilitate dispensing of the reagent layer composition into the electrode areas. The amount and type of surfactant is selected to assure the previously mentioned functions of the other chemical agents and to avoid a denaturing effect on the enzymes. The surfactant is selected from, but is not limited to, various anionic, cationic, non-ionic, and zwitterionic detergents. Examples of acceptable surfactants are polyoxyethylene ether, Tween 20, sodium cholate hydrate, hexadecylpyridinium cholide monohydrate and CHAPs. The preferred surfactant is a polyoxyethylene ether. More preferably, it is toctylphenoxypolyethoxyethanol and is available under the brand name Triton X-100. The concentration of Triton X-100 in the reagent layer composition is preferably about 0.01% (w/w) to about 2%.

Reagent Component

Suitable reagents include citric acid, phosphates, Tris, and the like. In the present invention, the pH of the reagent is preferably in the range from about 4.0 to about 9.0. Although other reagents are suitable for making a glucose-based biosensor, the preferred reagent in the present invention that provides the advantages of the present invention previously disclosed is selected from a phosphate reagent and a tris reagent. It is noted here and below that both the phosphate and the tris reagent in the reagent layer composition provide an unexpected result. It provides a glucose sensor with improved shelf life stability because each of these reagents allows the biosensor to function even when exposed to a high temperature environment for a relatively long period of time and/or when exposed to a high humidity environment for a relatively long time period.

Bulking Agent

An optional bulking agent that is water soluble and an inactive ingredient is preferably added into the reagent layer composition. The use of a bulking agent is advantageous when an electrode forming layer in the laminated body of a disposable biosensor is used to contain the reagent layer composition. The electrode openings in the electrode forming layer have a tendency to create and/or trap air bubbles/pockets when a sample fluid fills the capillary channel. The inclusion of an optional bulking agent in the reagent layer composition will reduce the tendency to trap bubbles at the electrode openings. Various sugars such as, for example, trehalose, galactose, sucrose, lactose, mannitol, mannose, fructose, lactitol, sorbitol, xylitol, nicotinamide, maltose, starch, and the like, may be added as a bulking agent into the reagent layer composition so long as they do not react with other ingredients and are inactive at the electrode surface. The bulking agent may be one chemical or a combination of chemicals. The amount of bulking agent in the reagent layer composition is in the range from about 1% to about 15% (w/w).

Although various reagent layer compositions are contemplated by the present invention that including containing only the basic, necessary chemical ingredients of an enzyme, an electron/redox mediator and a reagent component, the preferred reagent layer composition (referred to below as “reagent layer composition 1”) used for the glucose working electrode (W) contains Methocel 60 HG, polyethylene oxide, potassium ferricyanide, Triton X-100, bulking agents, FAD-GDH and tris reagent. The preferred reagent mixture (referred to below as “reagent layer composition 2”) used for the substrate/reference electrode (S) contains Methocel 60 HG, polyethylene oxide, potassium ferricyanide, Triton X-100, bulking agents, and tris reagent. The reagent layer composition 1 is used for the working electrode and the reagent layer composition 2 is used for the reference electrode. For simplicity, the reagent layer composition 2 is also used for the substrate electrode when a substrate electrode is included in the biosensor.

Preparation of the Preferred Reagent Layer Composition

The preferred reagent layer composition 1 is preferably prepared in two steps, although it may be prepared in one step:

Step 1: Into a tris reagent solution, add Methocel 60 HG, polyethylene oxide, fish gelatin, methylcellulose, bulking agent, and Triton X-100 in quantities within the ranges previously disclosed. Magnetically stir the solution until they are dissolved.

Step 2: Into the above solution, add an electron mediator, such as potassium ferricyanide, and a glucose related enzyme such as FAD-GDH in quantities within the ranges previously disclosed. Stir the solution until they are dissolved. The resulting solution is ready for application.

Reagent layer composition 2 is also preferably prepared in two steps although it too may be prepared in one step:

Step 1: Into a tris reagent solution, add Methocel 60 HG, polyethylene oxide, bulking agent, and Triton X-100 in quantities within the ranges previously disclosed. Magnetically stir the solution until they are dissolved.

Step 2: Into the above solution, add an electron/redox mediator such as potassium ferricyanide in a quantity within the ranges previously disclosed. Stir the solution until the electron/redox mediator is dissolved. The resulting solution is ready for application.

The chemical agents incorporated into the electrodes may also be done through the use of screen-printed techniques or the like. An organic carbon ink, which is known to those of ordinary skill in the respective art, is formed containing the glucose-related enzyme, the electron/redox mediator, the tris reagent and a polysaccharide binder.

Sensor Construction

Assembly of the various embodiments of the present invention is relatively straightforward. Generally for the 4-layer configuration, the base layer and reagent holding layer are laminated to each other followed by dispensing the appropriate reagent mixture into each of the reagent holding openings. After drying the reagent mixture, the channel forming layer is laminated onto the reagent holding layer and the cover is then laminated onto the channel forming layer. For the 3-layer construction, the base layer and the channel forming layer are laminated to each other followed by dispensing the appropriate reagent mixture as distinct drops/droplets into the U-shaped channel onto their respective electrically-conductive surface areas. After drying the reagent mixture, the cover is then laminated onto the channel forming layer.

More particularly for the 4-layer configuration shown in FIG. 1, a piece of a palladium or gold polyester film is cut to shape as illustrated in FIG. 2 forming base layer 20 of sensor 10. Even though mechanical scribing is an option, a laser is preferably used to score the conductive Pd or Au polyester film. As illustrated in FIG. 2, the film is scored by the laser such that three electrodes at sample fluid end 14 and three contact points 22, 24 and 26 are formed at electrical contact end 16. The scoring line is very thin but sufficient to create three separate and distinct electrically conductive paths. A scoring line 28 may optionally be made, but is not necessary, along the outer edge of base layer 20 to avoid potential static problems that could cause a noisy signal from the finished sensor 10. A piece of one-sided adhesive tape is then cut to size and shape, forming reagent holding layer 30 so that it will cover a major portion of conductive layer 21 of base layer 20 except for exposing a small electrical contact area illustrated in FIG. 1 by reference number 16.

Before attaching reagent holding layer 30 to base layer 20, three openings 32, 34 and 36 of substantially equal size are punched by laser, or by mechanical means such as a die-punch assembly, creating electrode openings 32, 34 and 36 in reagent holding layer 30. The shape of the electrode openings may be any shape. The preferred illustrated embodiment, the openings are circular. The preferred hole size for openings 32, 34 and 36 has a typical diameter of about 0.030 in. (0.76 mm) but may be any size. As illustrated in FIG. 2, electrode openings 32, 34 and 36 are aligned with each other and have a spacing of about 0.020 in. (0.508 mm) to about 0.050 in. (1.27 mm) between adjacent openings. The circular openings are for illustrative purposes only. It should be understood that the shape and size of the openings or the distance between them is not critical. The circular openings do not have to be substantially equal in size so long as the ratio of the surface areas remains substantially constant. Although the arrangement of the electrodes may be any combination, the preferred arrangement of the electrodes formed in openings 32, 34 and 36 is W (working electrode), R (reference electrode) and S (substrate electrode) as positioned from the sample inlet 18. Reagent holding layer 30 is then attached to base layer 20 in such a way as to define the electrode wells for creating working electrode W, reference electrode R and substrate electrode S.

Reagent layer composition 1 is dispensed into the working electrode well. As described above, reagent layer composition 1 is preferably a mixture of a bulking agent, a polymer binder, a surfactant, a redox mediator, enzyme, and a reagent solution but may be a mixture of only the important components, which are the polymer binder, the redox mediator, the enzyme, and the reagent solution. Similarly, reagent layer composition 2 is dispersed into the substrate electrode well and the reference electrode well. As mention above, the reagent layer composition 2 is similar to the reagent layer composition 1 but without the glucose-related enzyme.

After dispensing the reagents in their respective electrode areas, the reagents are dried at a temperature in the range of about room temperature to about 60° C. The length of time required to dry the reagents is dependent on the temperature at which the drying process is performed.

After drying, a piece of double-sided tape available from Adhesive Research or GIC is fashioned into chamber forming layer 40 containing a U-shaped channel 42. Chamber forming layer 40 is then layered onto reagent holding layer 30. As mentioned earlier, this chamber forming layer 40 serves as a spacer and defines the size of sample chamber 17.

A piece of a transparency film available from 3M or from GIC is fashioned into top layer 50. A vent opening 52 is made using the laser previously mentioned or by means of a die-punch. Vent opening 52 is located approximately 0.180 in. (4.57 mm) from fluid entrance 54. Top layer 50 is aligned and layered onto chamber forming layer 40 to complete the assembly of glucose biosensor 10, as illustrated in FIG. 1.

For the embodiment shown in FIG. 3, the assembly steps are similar except there is no “electrode forming layer” having specified openings to create wells into which the reagent layer compositions are dispensed. As described above, distinct drops/droplets of the reagent layer compositions are dispensed into the U-shaped channel onto their respective electrically conductive surface areas and then dried. It is also contemplated that only two electrically-conductive paths may be formed in the electrically-conductive layer 21 for forming only a working electrode and a reference electrode.

Testing the Glucose biosensor

When a fluid sample is applied to a single strip of the preferred embodiment of the present invention, the fluid sample enters the channel through the sampling inlet 18 and flows over electrodes W, R and S and stops at the threshold of the vent opening 52.

Chronoamperometry was used to measure the current response of the glucose biosensor using a Nova Glucose test meter. The glucose test strips made like those shown in FIG. 1 and described above were used to test the response of the biosensors of the present invention to the concentration of glucose. A blood sample containing a predefined amount of D-glucose is applied to the sample inlet 18 of the biosensor and enters the biosensor while a potential of 0.10 V to 0.70 volts (depending on reference electrode used) is applied between the working/substrate electrodes and the reference electrode. In the particular examples described here, the applied potential was 350 mV. The output response current is proportional to the concentration in the blood sample.

The chemical reaction that occurs in the biosensor is as follows. The D-glucose is oxidized into D-gluconic acid by the action of a glucose-based enzyme, which causes ferricyanide ions to be reduced to ferrocyanide ions. In the preferred embodiment, the glucose-based enzyme is FAD-GDH. The concentration of the ferrocyanide ions generated in the chemical reaction is proportional to the concentration of glucose in the sample solution and can be reoxidized at the electrode surface. Hence, based on the oxidation current generated by the ferrocyanide ions, the concentration of glucose is determined. In all of the tests conducted in the examples, a response current obtained in a reaction time of 3-10 seconds was used to calculate the concentration of glucose. A good linear relationship was observed between the response current and the glucose concentration, even in glucose concentrations higher than 900 mg/dL.

It is also noted that the biosensors of the present invention may optionally include the ability to make glucose measurements while compensating for the presence of hematocrit and/or interferents.

Some methods for measuring hematocrit (Hct) involve measuring blood resistivity (or conductivity) of a sample at a fixed frequency (See M. Maasrani et. al, Med. Biol. Eng. Comput., 11 (167-171) 1073, K. Cha, et. al., Physiol., Meas., 15 (129-137) 1994), or the change of impedance as a function of frequency (See C. G. Olthof et. al, Med. Biol. Eng. Comput., 32 (495-500) 1993). The unique feature of this invention involves measuring the current by applying a DC signal between the measuring electrode and the reference electrode to determine the concentration of the analyte, and also involves measuring the impedance by applying an AC signal between the measuring electrode and the reference electrode to determine the hematocrit concentration. The order of measuring the current and the impedance is not important. To measure the current using a DC signal, a fixed voltage is applied as the biasing potential between the measuring and reference electrodes using the electronics of the measuring meter. The electrochemical current produced by the electrochemical reactions described above is measured.

To measure impedance using an AC signal, the meter electronics generate a small sine wave or square wave of current superimposed upon the DC signal. The signal(s) produced is related to blood fluid impedance, which relates to the blood hematocrit concentration.

The effect of hematocrit is corrected by measuring the impedance (I) or resistance (R) or conductivity (C) of the blood samples. For purposes of the following equations, impedance and resistance is used interchangeably since the measurement value for the impedance or resistance is Ohms. Hematocrit effect can be corrected and eliminated using the following equations:

H=(a₁)(R²)+(a₂)(R)+a₃ (1)

where R is the resistance value measured in Ohms, H is the hematocrit value, and a₁, a₂ and a₃ are constants.

The hematocrit value is then used to mathematically correct the glucose concentration measured using the above described sensor. The following equation represents the calculation preformed using the calculated hematocrit level from equation (1):

C_{corr}=(Cₘₑₐ)*[b*H²+c*H+d] (2)

where C_{corr} is the corrected analytical concentration

Cₘₑₐ is the measured analytical concentration

b, c, and d are constants

H is the hematocrit level calculated from equation (1)

The constant values “a” through “d” above are empirically determined and depend on several factors such as the arrangement of the electrodes, the surface area of the electrodes, and the ratio of the surface areas of the electrodes. Once these factors are set, numerical analysis is performed on the test data obtained from predefined sample fluid standards using a plurality of biosensors having these preset factors in common. The correction can also be done by using the ohmic value without converting the ohmic value to hematocrit by simply substituting equation (1) into equation (2) and creating an algorithm to correct for the presence of hematocrit in the sample fluid.

For interferent correction, a biosensor electrode configuration is used in order to minimize the interference from oxidizable species, such as ascorbic acid, uric acid, acetaminophen, bilirubin, and other possible interferents present in the sample. This is achieved by incorporating a substrate electrode, which can be used to subtract a response current caused by those oxidizable species from the response current of the working electrode. In the embodiment where the surface area of the working electrode and the substrate electrode are substantially the same, the potential interference present in the sample fluid give relatively identical response signals from each of the substrate electrode and the working electrode. Thus, the difference in current response obtained in the blood sample is the current response from the glucose in the blood sample. In the embodiment where the working electrode and the substrate electrode are different sizes, so long as the ratio of the surface area of the working electrode to the surface area of the substrate electrode remains constant, a difference between the response signals knowing the ratio of the surface areas can be used to determine the current response resulting from the glucose in the blood sample.

The above described embodiments are based on amperometric analyses. Those skilled in the art, however, will recognize that a biosensor of the present invention may also utilize coulometric, potentiometric, voltammetric, and other electrochemical techniques to determine the concentration of an analyte in a sample.

The following examples illustrate the unique features of the present invention.

### Example 1

Biosensor stability was compared between biosensors with reagent layer compositions of the present invention incorporating different reagent solutions. The reagent layer compositions were disposed on gold electrodes formed on an insulating substrate. The biosensor with a reagent layer composition containing a citrate reagent is referred as sensor #1, a biosensor with a reagent layer composition containing a phosphate reagent is referred as sensor #2, and a biosensor with a reagent layer composition containing tris reagent is referred as sensor #3. The glucose biosensor vials containing a plurality of the above-described biosensors were left continually open to the room environment, which included a temperature of 25° C. and relative humidity of 50%. The biosensors' performance was checked using a glucose meter at the time intervals disclosed in Table 1 and measuring a glucose-containing control solution. The results are shown in Table 1.

TABLE 1A

| | 0 | 3 | 7 | 14 | 21 | 28 | 35 |
|---|---|---|---|---|---|---|---|
| Time | day | days | days | days | days | days | days |
| Sensor #1 | 113 | 112 | 118 | 121 | 133 | 137 | 146 |
| Sensor #2 | 114 | 113 | 117 | 118 | 119 | 122 | 127 |
| Sensor #3 | 113 | 112 | 112 | 113 | 116 | 114 | 113 |

The test data indicates that the biosensors with the citrate reagent showed an obvious increase in measurement values after being exposed for two weeks to the room temperature environment. The biosensors with the phosphate reagent showed an increase after about four weeks. The biosensors with the tris reagent were stable for at least three months (Data not shown beyond 35 days). It was unexpected to find that both the phosphate reagent containing biosensors and the tris reagent containing biosensors provided improved long term storage stability over comparable citrate reagent containing biosensors.

Additional biosensors of all three types described above were also exposed to a harsher environment. These additional biosensors were exposed to a temperature of 30° C. and relative humidity of 80%. The biosensors' performance was checked using a glucose meter at the time intervals disclosed in Table 1B using a glucose-containing control solution. The results are shown in Table 1B.

TABLE 1B

| Time | 0 | 1 | 3 | 5 | 9 | 15 | 25 | 35 |
|---|---|---|---|---|---|---|---|---|
| | day | day | days | days | days | days | days | days |
| Sensor #1 | 113 | 116 | 127 | NT | | | | |
| Sensor #2 | 114 | 113 | 118 | 123 | 129 | NT | | |
| Sensor #3 | 113 | 112 | 112 | 113 | 116 | 119 | 124 | NT |

NT: not trigger (i.e. will not wake up the meter to provide a reading)

The data indicates that the biosensors containing the citrate reagent in the reagent layer composition failed to work sometime between the third and the fifth day of exposure to the harsh conditions. The “NT” means that the meter did not display a reading for failure of the biosensor to provide a measured current when sample is applied to the strips. The biosensors containing the phosphate reagent in the reagent layer composition failed to work sometime between the ninth and fifteenth day of exposure to the harsh conditions. The biosensors containing the tris reagent in the reagent layer composition failed sometime between the twenty-fifth and the thirty-fifth day of exposure to the harsh conditions. It is clear from Table 1B that the biosensors containing the phosphate reagent and the tris reagent in the reagent layer composition are the most stable with better long term stability; up to seven times longer than comparable biosensors containing citrate reagent in the reagent layer composition. The biosensors with the citrate reagent in the reagent layer composition showed the least long term stability of the three biosensors in Table 1B.

### Example 2

In this example, a plurality of biosensors was made using different noble metals as the conductive electrode. This comparison was performed to determine if the type of noble metal used would also produce an unexpected result since all biosensor disclosures indicate that any noble metal may be used such as gold, platinum, palladium and the like as the underlying conductive electrode with no mention of any operational differences and/or advantages of one over the other. Again, the test involved exposing the biosensors to an extreme temperature.

The plurality of glucose biosensors containing tris reagent in the reagent layer composition on a palladium substrate is referred to as Sensor #4 and the plurality of glucose biosensors containing tris reagent in the reagent layer composition on a gold substrate is referred to as Sensor #5. Tris reagent was used in the reagent layer composition in this example since the biosensors whose reagent layer composition containing tris reagent from Example 1 showed the most long term stability. It is contemplated that the same effect as obtained below would be experienced by biosensors using other buffers in the reagent layer composition. The glucose biosensors were exposed to a high temperature environment of 50° C. The biosensors' performance was checked at the following time intervals of one week, one month, two months, three months, and four months in a glucose-containing control solution. The results are shown in Table 2A.

TABLE 2A
Stability at 50° C.

| Time | 0 | 1 week | 1 m | 2 m | 3 m | 4 m |
|---|---|---|---|---|---|---|
| Sensor #4 | 116 | 115 | 118 | 119 | 122 | 127 |
| Sensor #5 | 118 | 116 | 120 | 126 | 124 | 134 |

Both biosensors showed good stability at high temperature but the palladium substrate biosensors unexpectedly had better long term storage stability than the gold substrate biosensors.

### Example 3

In this example, various biosensors using the palladium substrate were made. One plurality of biosensors contained tris reagent in the reagent layer composition. Another plurality of biosensors contained phosphate reagent in the reagent layer composition. A third plurality of biosensors contained citrate reagent in the reagent layer composition.

Sensor #6 has the same composition as Sensor #4 in Example 2 above, which is a reagent layer composition containing tris reagent on a palladium substrate. Sensor #7 is a biosensor with a reagent layer composition containing phosphate reagent on a palladium substrate and Sensor #8 is a biosensor with a reagent layer composition containing citrate reagent on a palladium substrate. All biosensors of Sensor #6, Sensor #7 and Sensor #8 were exposed to room temperature environment (temperature of 25° C. and relative humidity of 50%). The biosensors' performance was tested at intervals of one month, two months, three months, four months, five months and six months. The results are shown in Table 3A.

TABLE 3A

| time | 0 | 2 w | 1 m | 2 m | 3 m | 4 m | 5 m | 6 m |
|---|---|---|---|---|---|---|---|---|
| Sensor #6 | 114 | 116 | 115 | 115 | 118 | 117 | 120 | 121 |
| Sensor #7 | 116 | 119 | 111 | 107 | NT | | | |
| Sensor #8 | 113 | 116 | 124 | NT | | | | |

NT: not trigger

From Table 3A, it is shown that the biosensors with a reagent layer composition containing the citrate reagent on the palladium substrate show poor long terms storage stability while the biosensors with a reagent layer composition containing the tris reagent on palladium substrate show the best long term storage stability.

It is further noted that the only difference in the biosensors used in the Table 3A tests and those in the Table 1A tests is the material of the electrically-conductive substrate used to make the working and reference electrodes. The aging conditions were the same, i.e. room temperature of 25° C. and relative humidity of 50%. In the Table 1A biosensors, the conductive substrate material is gold. In the Table 3A biosensors, the electrically-conductive substrate material is palladium. When compared, it is shown that the use of palladium instead of gold provides longer storage stability even for the citrate (from 2 weeks to over 1 month) and phosphate (from 4 weeks to over 2 months) buffer reagent layer compositions. For the tris reagent containing reagent layer composition, the long term storage stability increased from 3 months to over 6 months. These results were unexpected and significant. Thus, the effect of using palladium as the electrically-conductive substrate in a biosensor on the long term storage stability of biosensors was completely unexpected.

The stability of Sensor #6, Sensor #7 and Sensor #8 were also tested when the biosensor vials were left open and exposed to a harsher environment that included a temperature of 30° C. and relative humidity of 80%. The biosensors' performance was tested at intervals of one week, two weeks, one month, two months, and three months. The results are shown in Table 3B.

TABLE 3B

| Time | 0 | 1 w | 2 w | 1 m | 2 m | 3 m |
|---|---|---|---|---|---|---|
| Sensor #6 | 114 | 111 | 112 | 118 | 111 | 107 |
| Sensor #7 | 114 | 116 | 122 | NT | | |
| Sensor #8 | 114 | 124 | NT | | | |

NT: no trigger

From Table 3B, the biosensors with a reagent layer composition containing the citrate buffer on the palladium substrate show poor long term storage stability while the biosensors with a reagent layer composition containing the phosphate reagent on palladium substrate show better stability and those containing the tris reagent on the palladium substrate show the best stability.

It is further noted that the only difference in the biosensors used in the Table 3B tests and those in the Table 1B tests is the material of the conductive substrate used to make the working and reference electrodes. The aging conditions were the same, i.e. room temperature of 30° C. and relative humidity of 80%. In the Table 1B biosensors, the conductive substrate material is gold. In the Table 3B biosensors, the conductive substrate material is palladium. When compared, it is shown that the use of palladium instead of gold provides longer storage stability even for the citrate (from 3 days to over 1 week) and phosphate (from 9 days to over 2 weeks) buffer reagent layer compositions. Like the less harsh environment data described above, the effect of using palladium as the conductive substrate in a biosensor on the long term storage stability of biosensors was completely unexpected.

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

1. A disposable biosensor having improved long term storage stability, the biosensor comprising:
a working electrode and a reference electrode formed on an insulating substrate; and
a dissolvable reagent layer composition disposed directly on the working electrode wherein the reagent layer composition contains a glucose-based enzyme, a tris reagent selected from the group consisting of an acid of tris(hydroxymethyl)aminomethane and salts thereof, and a redox mediator.

2. The biosensor of claim 1 wherein the glucose-based enzyme is glucose dehydrogenase having flavin adenine dinucleotide as a coenzyme.

3. The biosensor of claim 1 wherein the glucose-based enzyme is selected from the group consisting of glucose oxidase, PQQ-glucose dehydrogenase, and NAD-glucose dehydrogenase.

4. The biosensor of claim 1 wherein the working electrode is made of gold, gold/tin oxide, platinum, or palladium.

5. The biosensor of claim 1 wherein the dissolvable reagent layer composition further includes a polymer binder.

6. The biosensor of claim 1 wherein the tris reagent is a tris(hydroxymethyl)aminomethane hydrochloride.

7. (canceled)

8. (canceled)

9. The biosensor of claim 1 wherein the tris reagent is selected from the group consisting of tris(hydroxymethyl)aminomethane hydrochloride, tris(hydroxymethyl)aminomethane acetate, tris(hydroxymethyl)aminomethane benzoate, tris(hydroxymethyl)aminomethane carbonate, tris(hydroxymethyl)aminomethane citrate, tris(hydroxymethyl)aminomethane maleate, tris(hydroxymethyl)aminomethane nitrate, tris(hydroxymethyl)aminomethane oxalate, tris(hydroxymethyl)aminomethane phosphate, tris(hydroxymethyl)aminomethane succinate, and tris(hydroxymethyl)aminomethane sulfate.

10. The biosensor of claim 1 further comprising a material selected from the group consisting of a surfactant and a bulking agent.

11. The biosensor of claim 10 wherein the glucose-based enzyme is selected from the group consisting of glucose oxidase, PQQ-glucose dehydrogenase, NAD-glucose dehydrogenase, and glucose dehydrogenase having flavin adenine dinucleotide as a coenzyme, the polymer binder is one or more selected from the group consisting of polyethylene oxide (PEO), polyethylene glycol, polyvinylpyrrolidone, starch, methylcellulose, hydroxypropylcellulose, polyvinyl alcohol (PVA), carboxy methyl cellulose (CMC), fish gelatin, and polyamino acids, the redox mediator is selected from the group consisting of potassium ferricyanide, potassium ferrocyanide, hexaamineruthenium (III) chloride, dimethylferrocene, ferricinium, 1,1′-ferrocene dicarboxylic, Meldola's blue, TCNQ, hydroquinone, dichlorophenoliondophenol, p-benoquinone, o-phenylenediamine, 3,4-dihydroxybenzaldehyde, and 1,10-phenanthroline quinine, the surfactant is selected from the group consisting of anionic, cationic, non-ionic, and zwitterionic detergents, and the bulking agent is one or more selected from the group consisting of trehalose, galactose, sucrose, lactose, mannitol, mannose, fructose, lactitol, sorbitol, xylitol, nicotinamide, maltose, and starch.

12. A reagent layer composition for use in a biosensor for amperometric detection of glucose in biological fluids wherein biosensor has an improved shelf life, the composition comprising:
a glucose-based enzyme;
a tris reagent selected from the group consisting of an acid of tris(hydroxymethyl)aminomethane and salts thereof; and
a redox mediator whereby the glucose-based enzyme reacts with a specific compound in a sample solution enabling the biosensor to measure a concentration of the specific compound in the sample.

13. The biosensor of claim 12 wherein the glucose-based enzyme is glucose dehydrogenase having flavin adenine dinucleotide as a coenzyme.

14. The biosensor of claim 12 wherein the glucose-based enzyme is selected from the group consisting of glucose oxidase, PQQ-glucose dehydrogenase, and NAD-glucose dehydrogenase.

15. The reagent layer composition of claim 12 further comprising a polymer binder.

16. The reagent layer composition of claim 12 wherein the tris reagent is tris(hydroxymethyl)aminomethane hydrochloride.

17. (canceled)

18. The reagent layer composition of claim 12 wherein the tris reagent is selected from the group consisting of tris(hydroxymethyl)aminomethane acetate, tris(hydroxymethyl)aminomethane benzoate, tris(hydroxymethyl)aminomethane carbonate, tris(hydroxymethyl)aminomethane citrate, tris(hydroxymethyl)aminomethane maleate, tris(hydroxymethyl)aminomethane nitrate, tris(hydroxymethyl)aminomethane oxalate, tris(hydroxymethyl)aminomethane phosphate, tris(hydroxymethyl)aminomethane succinate, and tris(hydroxymethyl)aminomethane sulfate.

19. The reagent layer composition of claim 12 further comprising one or more of a surfactant and a bulking agent.

20. The reagent layer composition of claim 19 wherein the glucose-based enzyme is selected from the group consisting of glucose oxidase, PQQ-glucose dehydrogenase, NAD-glucose dehydrogenase, and glucose dehydrogenase having flavin adenine dinucleotide as a coenzyme, the polymer binder is one or more selected from the group consisting of polyethylene oxide (PEO), polyethylene glycol, polyvinylpyrrolidone, starch, methylcellulose, hydroxypropylcellulose, polyvinyl alcohol (PVA), carboxy methyl cellulose (CMC), fish gelatin, and polyamino acids, the redox mediator is selected from the group consisting of potassium ferricyanide, potassium ferrocyanide, hexaamineruthenium (III) chloride, dimethylferrocene, ferricinium, 1,1′-ferrocene dicarboxylic, Meldola's blue, TCNQ, hydroquinone, dichlorophenoliondophenol, p-benoquinone, o-phenylenediamine, 3,4-dihydroxybenzaldehyde, and 1,10-phenanthroline quinine, the surfactant is selected from the group consisting of anionic, cationic, non-ionic, and zwitterionic detergents, and the bulking agent is one or more selected from the group consisting of trehalose, galactose, sucrose, lactose, mannitol, mannose, fructose, lactitol, sorbitol, xylitol, nicotinamide, maltose, and starch.

21. A disposable biosensor having improved shelf life, the biosensor comprising:
a working electrode and a reference electrode formed on an insulating substrate wherein the working electrode is made of gold, gold/tin oxide, platinum, or palladium; and
a dissolvable reagent layer composition disposed directly on the working electrode wherein the reagent layer composition contains a glucose-based enzyme, a tris reagent selected from the group consisting of an acid of tris(hydroxymethyl)aminomethane and salts thereof, and a redox mediator.

22. The disposable biosensor of claim 21 wherein the tris reagent is selected from the group consisting of tris(hydroxymethyl)aminomethane hydrochloride, tris(hydroxymethyl)aminomethane acetate, tris(hydroxymethyl)aminomethane benzoate, tris(hydroxymethyl)aminomethane carbonate, tris(hydroxymethyl)aminomethane citrate, tris(hydroxymethyl)aminomethane maleate, tris(hydroxymethyl)aminomethane nitrate, tris(hydroxymethyl)aminomethane oxalate, tris(hydroxymethyl)aminomethane phosphate, tris(hydroxymethyl)aminomethane succinate, and tris(hydroxymethyl)aminomethane sulfate.

23. The biosensor of claim 1 wherein the working electrode is made of palladium.

24. The biosensor of claim 21 wherein the working electrode is made of palladium.
